(19) United States
(12) Patent Application Publication
(10) Pub. No.: US 2017/0105633 A1
(43) Pub. Date: Apr. 20, 2017

(54) BIO-SIGNAL MEASURING APPARATUS WHICH OPERATES DIFFERENTLY ACCORDING TO TARGET

(71) Applicant: HUINNO CO., LTD., Seoul (KR)

(72) Inventor: Min-Yong Shin, Suwon (KR)

(21) Appl. No.: 15/393,881

(22) Filed: Dec. 29, 2016

Related U.S. Application Data
(63) Continuation of application No. PCT/KR2016/005690, filed on May 29, 2016.

(30) Foreign Application Priority Data
May 29, 2015 (KR) 1020150076646

(51) Int. Cl. A61B 5/0205; A61B 5/02; A61B 5/00; A61B 5/044; A61B 5/1455

(52) U.S. Cl. CPC A61B 5/0205; A61B 5/044; A61B 5/14551; A61B 5/6802; A61B 5/02007; A61B 5/7278; A61B 5/02416

(57) ABSTRACT

Provided herein are methods, systems, and apparatuses for measuring a bio-signal of a user. In one embodiment, a bio-signal measuring apparatus is provided that can operate by being combined with a plurality of target devices. For example, the bio-signal measuring apparatus can include an electrocardiogram (ECG) sensor. One or more PPG sensors can be included with a light emitting portion for generating light and a light receiving portion for receiving the light which is irradiated. The bio-signal measuring apparatus can be configured to measure one or more bio-signals using one or more of an ECG sensor and a PPG sensor, and the biological signal measuring apparatus can be configured to recognize the type of target device with which it is combined, activate a bio-signal measuring function, and correct bio-signal values.

This application is a continuation of International Application No. PCT/KR2016/005690 filed on May 30, 2016, which claims priority to Korean Application No. 10-2015-0076646 filed on May 29, 2015. The applications are expressly incorporated herein by reference.

### FIELD OF THE INVENTION

The present invention relates to measuring various biosignals of a user.

### BACKGROUND

Due to recent rapid progress in science and technology, the quality of life of all mankind is being enhanced and medical environment has changed a great deal. When a medical image is taken by means of X-ray, CT, fMRI or the like, it can take several hours or days to be able to interpret the image. However, a picture archive communication system (PACS) has been introduced to enable a medical image to be taken and then directly transmitted to a monitor screen of a radiology specialist for prompt interpretation thereof. Further, medical equipment for ubiquitous healthcare are also developed and widely spread so that self-checks on blood glucose and blood pressure are feasible at anytime and anywhere outside of a hospital.

For example, in the case of hypertension, which is one of the principal causes of various diseases and whose prevalence rate is increasing, there is a need for a monitoring system for consistent measurement and real-time notification of blood pressure.

One suggestion is a technique of applying ubiquitous healthcare (u-Health) to allow a patient suffering a chronic heart disease to visit a hospital significantly less frequently, by measuring blood pressure in real time by a blood pressure measurement sensor inserted in a pulmonary artery of the patient, and then wirelessly transmitting the measured blood pressure to an attending doctor so that the doctor may monitor variations in the blood pressure in the pulmonary artery of the patient and give a prescription to the patient. While this approach has the advantage of measuring blood pressure continuously and accurately, it is implemented through an invasive method and thus can cause operational difficulties and risks of arterial damage, infection, and similar issues. Accordingly, this technique is only used when a medical necessary.

It is thus desirable to find methods for non-invasively measuring blood pressure in real time without inserting any blood pressure measurement sensor in arterial blood vessels and monitoring blood pressure in a ubiquitous environment and then providing biofeedback on the measured blood pressure to a user so that the user may take steps to adjust the blood pressure.

However many biosignal measurement methods can be performed only by specific devices provided with sensor modules capable of measuring biosignals. Therefore, a user can be limited to measuring biosignals only when the user carries such a specific device.

### SUMMARY

One approach to address this issue is a method of estimating blood pressure of a user in real time based on biosignals such as electrocardiogram (ECG), photoplethysmogram (PPG) and oxygen saturation level (SpO₂) signals, which are measured by an ECG sensor module and a PPG sensor module (a light sensor module) provided in a wearable device.

As applied below, an electrocardiogram (ECG) is a waveform consisting of a vector sum of action potentials generated by a special excitatory and conductive system of a heart. For example, it can be obtained by measuring, from an electrode contacting the outside of a body, a signal corresponding to a vector sum of active potentials generated by the components of the heart such as sinoatrial node (SA node), atrioventricular node (AV node), His bundle, His bundle branch, and Purkinje fibers. For example, the ECG signal may be obtained using a method such as a standard limb lead method, based on signals measured by a plurality of electrodes constituting the ECG sensor module.

A photoplethysmogram (PPG) signal is a pulse wave signal measured at peripheral blood vessels when blood ejected during a ventricular systole is delivered to the peripheral blood vessels. The PPG signal can be measured using optical properties of biological tissues. For example, it can be obtained by attaching a PPG sensor module (a light sensor module) capable of measuring a pulse wave signal to a region where peripheral blood vessels are distributed (e.g., fingertips or the tips of toes) and then converting variations in blood stream flow (corresponding to variations in the volume of the peripheral blood vessels) into variations in light intensity. Meanwhile, rather than using only the PPG signal, a correlation between the PPG and ECG signals can be analyzed to derive information such as a pulse transit time (PTT) or a pulse wave velocity (PWV) for use in, for example, diagnosing cardiovascular diseases. For example, feature points are obtained from a second derivative of a PPG signal and time intervals are measured with respect to peak points (or R waves) of an ECG signal to derive PTT and PWV signals for use in diagnosing blood vessel conditions, artery hardening, peripheral circulatory disturbance, and the like.

An oxygen saturation level (or saturation of peripheral oxygen; SpO₂) signal is a biosignal representing the content of oxygen present in hemoglobin among various components of blood. The SpO₂ signal can be measured by emitting red light and infrared light through a PPG sensor module (a light sensor module) in alternating periods so that the emitted light is irradiated to peripheral blood vessels of a body part, and then observing variations in the intensity of light reflected from the body part and received by a light receiving unit.

Provided herein is a biosignal measurement apparatus operable in combination with various counterpart objects so that a user may more easily measure his/her biosignals at a desired point of time.

Also provided herein is a method of more effectively operating the biosignal measurement apparatus by recognizing a type of the counterpart object combined with the biosignal measurement apparatus and activating necessary functions according to the type of the combined counterpart object.

Provided herein is another method to measure a biosignal more accurately by specifying a body part from which the measured biosignal is generated according to the type of the combined counterpart object, and accordingly correcting the measured biosignal.

The objects of the invention are not limited to the aforementioned ones, and other objects not mentioned herein will be apparently appreciated by those skilled in the art.

According to one embodiment, a biosignal measurement apparatus can be provided that is operable in combination with a plurality of counterpart objects. The biosignal measurement apparatus can include an ECG sensor or sensor module with a first electrode formed on a rear side thereof and a second electrode formed apart from the first electrode, and at least one PPG sensor or sensor module including a light emitter or emitting unit for generating light to be irradiated to a body part, and a light receiver or receiving unit for receiving light irradiated by the light emitter or emitting unit and reflected by the body part. The biosignal measurement apparatus can be configured to measure at least one of electrocardiogram (ECG), photoplethysmogram (PPG) and oxygen saturation level (SpO₂) biosignals of a user using at least one of the ECG sensor or sensor module and the PPG sensor or sensor module. Further, the biosignal measurement apparatus can be configured to recognize a type of the combined counterpart object, to activate functions for measuring measurable biosignals according to the type of the counterpart object, and to correct values of the measured biosignals according to the type of the combined counterpart object.

According to one embodiment provided herein, the biosignal measurement apparatus can include a display or display module formed on a front side of the biosignal measurement apparatus to display information to the user, and the display or display module can be provided with a measurement area for measuring biosignals of the user. Red (R) subpixels for forming red light and infrared (IR) subpixels for forming infrared light can be included in a pixel structure formed in the measurement area of the display or display module. The red (R) subpixels and infrared (IR) subpixels formed in the measurement area of the display or display module may form the light emitter or emitting unit of one of the at least one PPG sensor or sensor module. Further, the light receiver or receiving unit of the PPG sensor or sensor module whose light emitter or emitting unit is formed with the red (R) subpixels and infrared (IR) subpixels can be formed in the measurement area of the display or display module.

According to another embodiment provided herein, the biosignal measurement apparatus can be further provided with at least one additional electrode for the ECG sensor or sensor module spaced apart from the first electrode and the second electrode.

According to an embodiment, the biosignal measurement apparatus can be operable as mounted on a watch-type counterpart object. When the biosignal measurement apparatus is mounted on the watch-type counterpart object, the biosignal measurement apparatus can be configured to activate a function for measuring an ECG signal by the ECG sensor or sensor module and a function for measuring PPG and SpO₂ signals by the PPG sensor or sensor module.

According to another embodiment, when the biosignal measurement apparatus is mounted on the watch-type counterpart object, the biosignal measurement apparatus can be configured to correct the ECG signal on the assumption that the ECG signal is measured while the first electrode A is in contact with the user's wrist and the second electrode B is in contact with the user's finger. The biosignal measurement apparatus can also be configured to further perform a function for estimating blood pressure of the user based on the measured ECG, PPG and SpO₂ signals.

According to one embodiment, the biosignal measurement apparatus can be operable as mounted on a necklace-type counterpart object. When the biosignal measurement apparatus is mounted on the necklace-type counterpart object, the biosignal measurement apparatus can be configured to activate a function for measuring an ECG signal by the ECG sensor or sensor module and a function for measuring PPG and SpO₂ signals by the PPG sensor or sensor module.

According to another embodiment, when the biosignal measurement apparatus is mounted on the necklace-type counterpart object, the biosignal measurement apparatus can be configured to correct the ECG signal on the assumption that the ECG signal is measured while the first electrode and the second electrode are in contact with different fingers of the user. The biosignal measurement apparatus can be configured to further perform a function for estimating blood pressure of the user based on the measured ECG, PPG and SpO₂ signals.

According to an embodiment, at least one auxiliary electrode can be further provided on a necklace string of the necklace-type counterpart object that can be combined with the biosignal measurement apparatus, and the biosignal measurement apparatus can be configured to measure an ECG signal of the user based on signals from at least two electrodes contacting the user's body, among the first electrode and the second electrode formed in the biosignal measurement apparatus and the at least one auxiliary electrode formed on the necklace string of the necklace-type counterpart object. In this case, the biosignal measurement apparatus can be configured to correct the ECG signal on the assumption that signals from the auxiliary electrode formed on the necklace string are generated while the auxiliary electrode is in contact with the user's neck, and those from the first electrode and the second electrode of the biosignal measurement apparatus are generated while the first electrode and the second electrode are in contact with the user's fingers.

According to another embodiment, the biosignal measurement apparatus can be operable as mounted on a vehicle steering wheel. When the biosignal measurement apparatus is mounted on the vehicle steering wheel, the ECG sensor or sensor module and the PPG sensor or sensor module formed in the biosignal measurement apparatus are deactivated, and a function for measuring an ECG signal by an ECG sensor or sensor module for the steering wheel, which includes a first switching electrode and a second switching electrode formed in the vehicle steering wheel, and a function for measuring PPG and SpO₂ signals by a PPG sensor or sensor module for the steering wheel, which is formed in the vehicle steering wheel, are activated.

According to one embodiment, when the biosignal measurement apparatus is mounted on the vehicle steering wheel, the biosignal measurement apparatus can be configured to correct the ECG signal on the assumption that the ECG signal is measured while the first switching electrode and the second switching electrode are in contact with different fingers of the user. The biosignal measurement apparatus can be configured to further perform a function for estimating blood pressure of the user based on the measured ECG, PPG and SpO₂ signals.

According to another embodiment, the biosignal measurement apparatus is operable as mounted on a finger rest of an oxygen saturation level measurement device. When the biosignal measurement apparatus is mounted on the finger rest of the oxygen saturation level measurement device, the biosignal measurement apparatus can be configured to activate a function for measuring a SpO₂ signal by the PPG sensor or sensor module.

According to an embodiment, when a plurality of electrodes of the ECG sensor or sensor module formed in the biosignal measurement apparatus are put into contact with the user's body while the biosignal measurement apparatus is mounted on the finger rest of the oxygen saturation measurement device, the biosignal measurement apparatus can be configured to further activate a function for measuring an ECG signal by the ECG sensor or sensor module.

In addition, the biosignal measurement apparatus can further include other configurations without departing from the technical ideas of the invention.

The biosignal measurement apparatus can be configured to measure a variety of biosignal information (ECG, PPG, SpO₂, etc.) generated from a user's body in combination with various counterpart objects so that the user may more easily measure his/her biosignals at a desired point of time.

The biosignal measurement apparatus can also be configured to recognize a type of the combined counterpart object and activate only necessary biosignal measurement functions according to the type of the combined counterpart object so that the biosignal measurement apparatus may be operated more effectively.

A biosignal can be measured more accurately because the biosignal measurement apparatus can be configured to specify a body part from which the measured biosignal is generated according to the type of the combined counterpart object, and accordingly correct the measured biosignal.

### BRIEF DESCRIPTION OF THE DRAWINGS

FIG. 1 schematically shows a front perspective view of a biosignal measurement apparatus according to one embodiment.

FIG. 2 schematically shows a rear perspective view of the biosignal measurement apparatus shown in FIG. 1.

FIG. 3 illustratively shows the configuration of a biosignal measurement apparatus according to another embodiment.

FIG. 4 shows one embodiment of a counterpart object (a watch-type counterpart object) that can be combined with a biosignal measurement apparatus.

FIG. 5 shows that the biosignal measurement apparatus can be combined with the watch-type counterpart object shown in FIG. 4.

FIG. 6 shows another embodiment of a counterpart object (a necklace-type counterpart object) that can be combined with a biosignal measurement apparatus.

FIG. 7 shows that the biosignal measurement apparatus can be combined with the necklace-type counterpart object shown in FIG. 6.

FIG. 8 shows another embodiment of a necklace-type counterpart object that can be combined with a biosignal measurement apparatus.

FIG. 9 shows an embodiment of a counterpart object (a vehicle steering wheel) that can be combined with a biosignal measurement apparatus.

FIG. 10 shows that the biosignal measurement apparatus described herein can be combined with the vehicle steering wheel shown in FIG. 9.

FIG. 11 shows still another embodiment of a counterpart object (a finger rest of an oxygen saturation level measurement device) that can be combined with a biosignal measurement apparatus.

FIG. 12 shows that the biosignal measurement apparatus can be combined with the finger rest of the oxygen saturation level measurement device shown in FIG. 11.

### DETAILED DESCRIPTION

Hereinafter, various embodiments will be described in detail with reference to the accompanying drawings to enable those skilled in the art to easily implement the embodiments.

In order to clearly illustrate the embodiments, detailed descriptions on the elements irrelevant to the illustrations will be omitted, and the same elements will be denoted by the same reference numerals throughout the entire specification. Further, the shape and size of each element shown in the drawings are arbitrarily shown for convenience of illustration, and the present invention is not necessarily limited to the shown shape and size. That is, specific shapes, structures and characteristics described herein may be implemented as modified from one embodiment to another without departing from the spirit and scope of the invention. Furthermore, it shall be understood that the locations or arrangements of individual elements may also be modified without departing from the spirit and scope of the invention. Therefore, the following detailed description is not to be taken in a limiting sense, and the scope of the invention is to be taken as encompassing the scope of the appended claims and all equivalents thereof.

Biosignal Measurement Apparatus

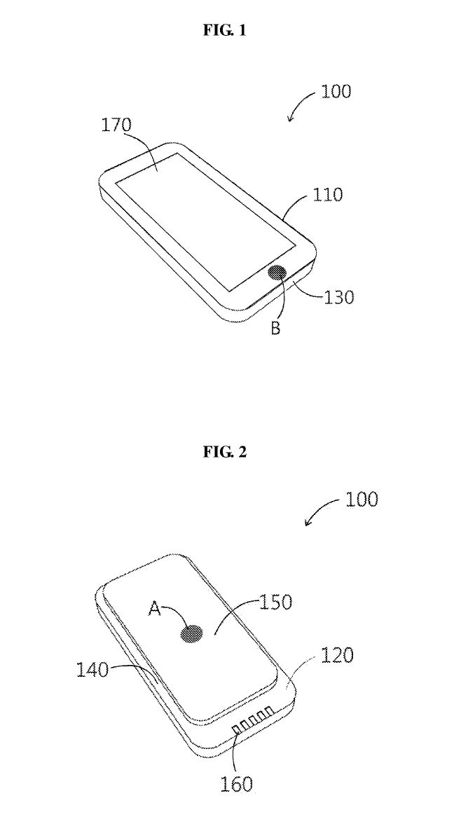

FIGS. 1 and 2 illustratively show a biosignal measurement apparatus 100 according to one embodiment. FIG. 1 shows a front perspective view of the biosignal measurement apparatus 100 according to one embodiment, and FIG. 2 shows a rear perspective view of the biosignal measurement apparatus 100 according to one embodiment. The biosignal measurement apparatus of the embodiment shown in FIG. 1 can include a front side 110 with a display or display screen of a display module, a rear side 120 disposed opposite to the front side 110, and a lateral side 130 connecting the front side 110 and the rear side 120. The central part of the rear side 120 can be configured to protrude through a stepped part 140, so that a part (such as protrusion 150) of the rear side of the biosignal measurement apparatus 100 can be outwardly exposed when the biosignal measurement apparatus 100 is mounted on a counterpart object to be described below.

An electrical contact 160 can be formed on the rear side 120 of the biosignal measurement apparatus 100 and can be configured to provide an electrical connection with a counterpart object when the biosignal measurement apparatus 100 is mounted on the counterpart object (for example, see FIG. 2). When the biosignal measurement apparatus 100 is mounted on the counterpart object, the electrical contact 160 can contact an electrical contact formed in the counterpart object to form an electrical connection with the counterpart object. In the embodiment shown in FIG. 2, an electrical contact can be formed to form an electrical connection between the biosignal measurement apparatus 100 and the counterpart object. However, it is also possible to form an electrical connection between the biosignal measurement apparatus 100 and the counterpart object through a wireless connection using infrared light (RF), Bluetooth or the like, instead of a physical connection through the electrical contact.

The biosignal measurement apparatus 100 can be configured to measure various biosignals of a user with sensors and/or sensor modules (such as ECG sensors or sensor modules, PPG sensors or sensor modules, etc.) formed in the main body of the apparatus.

Specifically, the biosignal measurement apparatus 100 of the embodiment shown in FIGS. 1 and 2 can be configured such that a first electrode A configured to measure biosignals can be formed on the rear side, and a second electrode B configured to measure biosignals can be formed where the first electrode A is not formed (e.g., on the front side 110 or the lateral side 130 of the main body). For example, FIG. 1 illustratively shows an embodiment in which the second electrode B can be formed on the front side 110 of the main body. The first electrode A and the second electrode B formed in the main body of the apparatus can form an ECG sensor or sensor module for measuring an ECG signal of a user. For example, when a user puts a body part into contact with the second electrode B while another body part is in contact with the first electrode A, a signal related to an electrocardiogram (ECG) of the user's body can be measured by the first electrode A and the second electrode B.

Meanwhile, the second electrode B can also be disposed on the display or display screen of the display module 170 formed on the front side of the biosignal measurement apparatus 100. When the second electrode B is disposed on the display or the display screen constituting the display module of the biosignal measurement apparatus 100, a user can put a body part into contact with the display (or the second electrode B) while another body part of the user is in contact with the first electrode A, so that an ECG signal may be measured.

In the embodiment shown in FIGS. 1 and 2, an ECG sensor or sensor module configured to measure a signal related to an ECG of a user can include a plurality of electrodes, such as two electrodes (the first electrode A and the second electrode B) that can be formed apart from each other. However, the ECG sensor can also be formed with three electrodes by further forming another electrode (the third electrode) spaced apart from the two electrodes. Further, the ECG sensor can include four or more electrodes by further providing additional electrodes spaced apart from the other electrodes.

Further, the biosignal measurement apparatus 100 can include at least one PPG sensor or sensor module (such as a light sensor or sensor module) configured to measure PPG and/or SpO₂ signals. As described above, the PPG and SpO₂ signals can be measured by irradiating light generated by a light emitter or emitting unit of the PPG sensor or sensor module (light sensor or sensor module) to a tip of a user's hand or foot, and then observing variations in the intensity of light transmitted or reflected by the user's body and received by a light receiver or receiving unit. Although the mounting position of the PPG sensor is not particularly limited, the PPG sensor can be formed together where the electrodes constituting the ECG sensor are formed. When the PPG sensor is formed together where the electrodes constituting the ECG sensor are formed, the PPG and SpO₂ signals can be measured while the ECG signal of the body is measured.

The PPG sensor for measuring PPG and/or SpO₂ signals can include a light emitter or emitting unit (not shown) including a red LED configured to generate red light having a wavelength of about 660 nm and an infrared LED configured to generate infrared light having a wavelength of about 940 nm, and a light receiver or receiving unit (not shown) including a photo diode and/or a photo transistor. For example, the biosignal measurement apparatus 100 according to one embodiment can include a PPG sensor or sensor module that can be provided where the first electrode A is formed and that can include a light emitter or emitting unit with an infrared LED and a light receiver or receiving unit with a photo diode.

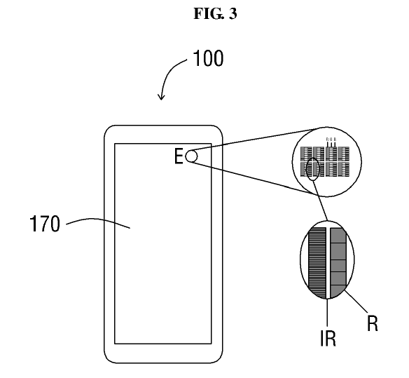

Meanwhile, the PPG sensor for measuring PPG and/or SpO₂ signals can also be implemented using the display or display module 170 formed on the front side of the apparatus. FIG. 3 illustratively shows an embodiment in which the PPG sensor is implemented using the display 170 of the biosignal measurement apparatus 100.

For example, the biosignal measurement apparatus 100 can be provided with a measurement area E for measuring biosignals (PPG and/or SpO₂ signals) of a user at a part of the display, as shown in FIG. 3. As described above, red light can be irradiated to a human body in order to measure a PPG signal, and red light and infrared light can be irradiated to a human body in order to measure a SpO₂ signal. To this end, the biosignal measurement apparatus 100 can be configured such that infrared (IR) subpixels for forming infrared light are further included in a pixel structure of the measurement area E of the display, in addition to commonly used RGB subpixels (i.e., red (R) subpixels for forming red light, green (G) subpixels for forming green light, and blue (B) subpixels for forming blue light), as shown in FIG. 3. According to this configuration, red light and infrared light may be irradiated to the photographing area E by the red (R) subpixels and infrared (IR) subpixels included in the pixel structure of the measurement area E of the display, and the red light and infrared light irradiated to the photographing area E may function as a light emitter of the PPG sensor (light sensor) for measuring PPG and/or SpO₂ signals. Further, the measurement area E of the display can be further provided with a light receiver for receiving light irradiated by the red (R) subpixels and infrared (IR) subpixels and reflected by the human body. The use of the biosignal measurement apparatus 100 configured as above enables biosignals such as PPG and/or SpO₂ signals to be measured by the display of the biosignal measurement apparatus 100, without forming additional light sensors in the biosignal measurement apparatus 100.

The use of the biosignal measurement apparatus 100 configured as above enables various biosignals (e.g., ECG, PPG and SpO₂ signals) of a user to be measured by the sensors (e.g., the ECG sensor, the PPG sensor, etc.) provided in the biosignal measurement apparatus 100. Further, information on the measured biosignals can be stored in a storage unit (not shown) provided in the biosignal measurement apparatus 100, or may be analyzed and processed by a controller or control unit (not shown) of the biosignal measurement apparatus 100. For example, it is possible to estimate blood pressure of the user in real time using the ECG, PPG and SpO₂ signals measured by the ECG sensor and the PPG sensor described as above. In connection with the details of how to measure and analyze biosignals and to estimate blood pressure based on the measured biosignals, reference may be made to the disclosures of Korean Patent Application Nos. 2013-116158 and 2012-54770 of the inventor, which are incorporated herein by reference in their entirety.

Counterpart Objects that can be Combined with the Biosignal Measurement Apparatus

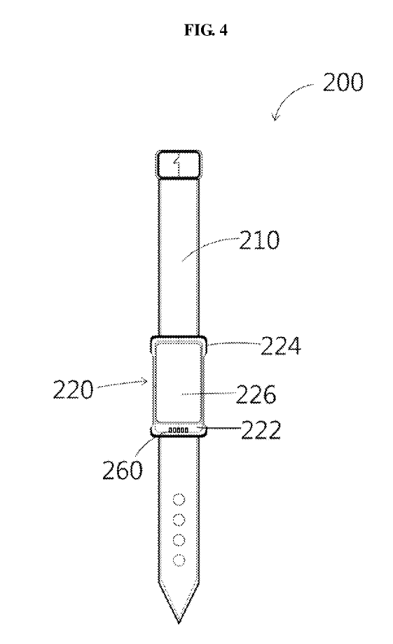

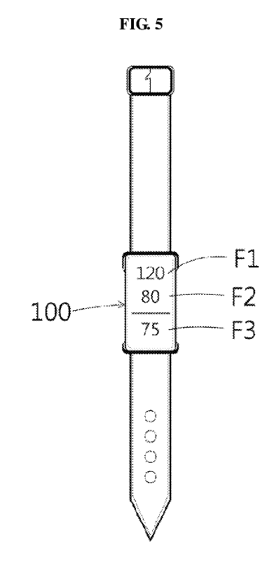

Next, FIGS. 4 to 11 illustrate embodiments in which the biosignal measurement apparatus 100 is operated as mounted on various counterpart objects.

[Watch-Type Counterpart Object]

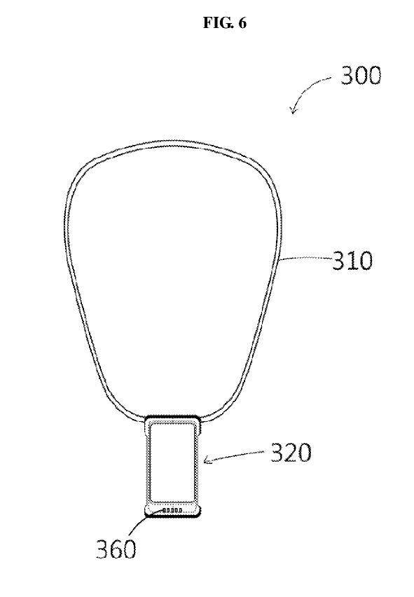

For example, FIGS. 4 and 5 show an embodiment in which the biosignal measurement apparatus 100 is operated as mounted on a watch-type counterpart object 200. As shown in FIG. 4, the watch-type counterpart object 200 can include a band part 210 configured to wrap around a user's wrist, and a mounting part 220 configured to mount the biosignal measurement apparatus 100. The mounting part 220 can be formed in a shape corresponding to the external shape of the biosignal measurement apparatus 100, and can be configured to hold the biosignal measurement apparatus 100. For example, the mounting part 220 can generally include a base part 222 and a wall part 224 extending substantially vertically from the base part, so that the inner space formed by the base part 222 and the wall part 224 can accommodate the biosignal measurement apparatus 100. The structure of the mounting part 220 is not limited to the shape shown in FIG. 4, and can be formed in any of various structures capable of holding and stably supporting the biosignal measurement apparatus 100. Meanwhile, a through hole 226 to which the protrusion 150 formed on the rear side of the biosignal measurement apparatus 100 can be inserted can be formed at the center of the base part 222 of the mounting part 220, so that the rear side of the biosignal measurement apparatus 100 can be outwardly exposed when the biosignal measurement apparatus 100 is mounted on the watch-type counterpart object 200. Further, on the inner side of the base part 222 of the mounting part 220, an electrical contact 260 that is electrically connected to the electrical contact 160 of the biosignal measurement apparatus 100 can be provided where the electrical contact 160 of the biosignal measurement apparatus 100 is placed when the biosignal measurement apparatus 100 is mounted. By an electrical connection between the electrical contact 160 of the biosignal measurement apparatus 100 and the electrical contact 260 of the watch-type counterpart object 200, the biosignal measurement apparatus 100 can recognize the type of the combined counterpart object 200 and accordingly activate necessary biosignal measurement functions.

FIG. 5 shows that the biosignal measurement apparatus 100 can be combined with the watch-type counterpart object 200 shown in FIG. 4. As shown in FIG. 5, when the biosignal measurement apparatus 100 is mounted on the watch-type counterpart object 200, an ECG signal of a user can be measured by the ECG sensor provided in the biosignal measurement apparatus 100, and PPG and SpO₂ signals can be measured by the PPG sensor. Further, it is also possible to estimate blood pressure of the user in real time using information on the ECG, PPG and SpO₂ signals. Thus, when the biosignal measurement apparatus 100 according to one embodiment is mounted on the watch-type counterpart object 200 through the electrical contact 160, the biosignal measurement apparatus 100 can be configured to activate a function for measuring an ECG signal by the ECG sensor, a function for measuring PPG and/or SpO₂ signals by the PPG sensor, and a function for estimating blood pressure in real time based on these biosignals.

Specifically, as shown in FIG. 5, when the watch-type counterpart object 200 on which the biosignal measurement apparatus 100 is mounted is worn on a user's wrist, the first electrode A formed on the rear side of the biosignal measurement apparatus 100 is always in contact with the user's wrist. In this state, when the user puts a finger of the other hand into contact with the second electrode B formed in the biosignal measurement apparatus 100 (e.g., the display on the front side of the biosignal measurement apparatus 100), an ECG signal of the user can be measured by the first electrode A and the second electrode B. Further, PPG and SpO₂ signals of the user can be measured by the PPG sensor formed in the biosignal measurement apparatus 100 (e.g., the measurement area E formed on the display 170 in the embodiment shown in FIG. 3). It is possible to estimate blood pressure of the user in real time using these biosignals.

Meanwhile, when the biosignal measurement apparatus 100 is used as mounted on the watch-type counterpart object 200 shown in FIGS. 4 and 5, biosignals (e.g., ECG signal) can be measured while the first electrode A is in contact with the user's wrist and the second electrode B is in contact with the user's finger. In general, the biosignals indicate different values depending on the body parts contacting the sensors. Thus, in order to acquire more accurate biosignal information, it is helpful and can be necessary in certain embodiments to specify the body part from which the measured biosignal is generated and accordingly correct the measured biosignal. To this end, when it is recognized that the biosignal measurement apparatus 100 is combined with the watch-type counterpart object 200 through the electrical contact 160, the biosignal measurement apparatus 100 can be configured to assume that an ECG signal is measured while the user's wrist is in contact with the first electrode A and the user's finger is in contact with the second electrode B, and to accordingly correct the biosignal (ECG signal). In some configurations, the body part in which the biosignal is generated can be specified more accurately so that the biosignal can be acquired more accurately.

The information on the measured and/or estimated biosignals can be provided to the user through the display or display screen of the display module formed on the front side of the biosignal measurement apparatus 100 (for example, information on numerical values of systolic blood pressure F1, diastolic blood pressure F2, pulse F3 and the like can be displayed on the display 170 as shown in FIG. 5), or may be stored in a storage unit (not shown) provided in the biosignal measurement apparatus.

[Necklace-Type Counterpart Object]

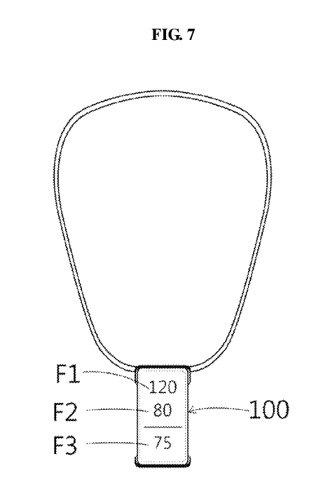

FIGS. 6 to 8 illustrate an embodiment in which the biosignal measurement apparatus 100 can be operated as combined with a necklace-type counterpart object 300. As shown in FIG. 7, the necklace-type counterpart object 300 can include a necklace string 310 configured to hang around a user's neck, and a mounting part 320 connected to the necklace string and configured to hold the biosignal measurement apparatus 100. The mounting part 320 can be formed in a shape similar to that of the mounting part 220 of the watch-type counterpart object 200 shown in FIG. 4, and an electrical contact 360 that is electrically connected to the electrical contact 160 of the biosignal measurement apparatus can be provided on the inner side of the mounting part.

FIG. 6 shows that the biosignal measurement apparatus 100 can be mounted on the necklace-type counterpart object 300 shown in FIG. 5. As shown in FIG. 6, when the biosignal measurement apparatus 100 is mounted on the necklace-type counterpart object 300, an ECG signal of a user can be measured by the first electrode A and the second electrode B formed in the biosignal measurement apparatus 100, and PPG and SpO₂ signals can be measured by the PPG sensor. Further, it is possible to estimate blood pressure of the user in real time using these signals. Thus, when it is recognized that the biosignal measurement apparatus 100 according to one embodiment is mounted on the necklace-type counterpart object 300 through the electrical contact 160, the biosignal measurement apparatus 100 can be configured to activate a function for measuring an ECG signal by the ECG sensor, a function for measuring PPG and/or SpO₂ signals by the PPG sensor, and a function for estimating blood pressure in real time based on these biosignals.

Specifically, as shown in FIG. 7, when the user puts a finger into contact with the first electrode A formed on the rear side of the biosignal measurement apparatus 100 and puts another finger into contact with the second electrode B (e.g., the display of the biosignal measurement apparatus 100) while the biosignal measurement apparatus 100 is mounted on the necklace-type counterpart object 300, an ECG signal of the user is measured by the first electrode A and the second electrode B. Further, PPG and SpO₂ signals of the user are measured by the PPG sensor formed in the biosignal measurement apparatus 100 (e.g., the measurement area E formed on the display 170 in the embodiment shown in FIG. 3). It is possible to estimate blood pressure of the user in real time using these biosignals.

Meanwhile, in the case of the watch-type counterpart object 200 shown in FIGS. 4 and 5, biosignals (ECG signal) are generally measured while the first electrode A is in contact with the user's wrist and the second electrode B is in contact with the user's finger. However, in the case of the necklace-type counterpart object 300 shown in FIGS. 6 and 7, biosignals (ECG signal) can be generally measured while both of the first electrode A and the second electrode B are in contact with the user's fingers. Thus, when it is recognized that the biosignal measurement apparatus 100 according to one embodiment is mounted on the necklace-type counterpart object 300 through the electrical contact 160, the biosignal measurement apparatus 100 can be configured to assume that an ECG signal is measured while a finger of the user is in contact with the first electrode A and another finger of the user is in contact with the second electrode B, and to accordingly correct the ECG signal based on this assumption so that the biosignal can be measured more accurately.

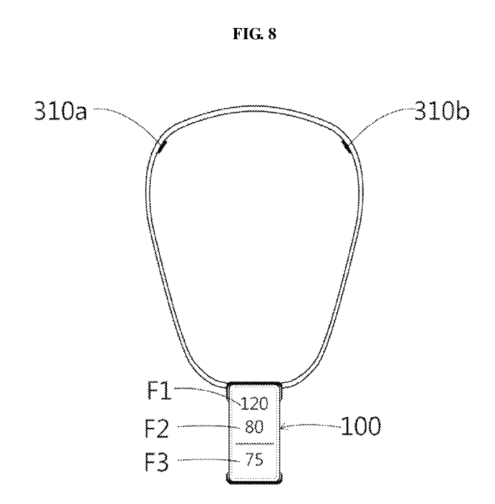

Further, the necklace-type counterpart object 300, which may be combined with the biosignal measurement apparatus 100, can be further provided with auxiliary electrodes that can be used for measuring an ECG signal. FIG. 8 shows an embodiment in which a first auxiliary electrode 310a and a second auxiliary electrode 310b are formed on the necklace string 310 of the necklace-type counterpart object 300. According to this configuration, it is possible to measure an ECG signal of the user by at least two electrodes contacting the user's body, among the first electrode A and the second electrode B formed in the biosignal measurement apparatus 100 and the auxiliary electrodes formed in the necklace-type counterpart object 300. For example, the ECG signal can be measured while the first auxiliary electrode 310a and the second auxiliary electrode 310b formed on the necklace string 310 of the necklace-type counterpart object 300 are in contact with the user's neck, or while at least one of the auxiliary electrodes formed in the necklace-type counterpart object 300 is in contact with the user's neck and at least one of the first electrode A and the second electrode B of the biosignal measurement apparatus 100 is in contact with the user's finger. Here, the auxiliary electrodes formed on the necklace string 310 of the necklace-type counterpart object 300 can be generally in contact with the user's neck, while the electrodes formed in the biosignal measurement apparatus 100 can be generally in contact with the user's fingers. Thus, the biosignal measurement apparatus 100 according to one embodiment can be configured to assume that signals from the auxiliary electrodes 310a, 310b formed on the necklace string 310 are generated while the auxiliary electrodes 310a, 310b are in contact with the user's neck, and those from the first electrode A and the second electrode B of the biosignal measurement apparatus 100 are generated while the first electrode A and the second electrode B are in contact with the user's fingers, and to accordingly correct the ECG signal based on these assumptions so that the biosignal can be measured more accurately.

Meanwhile, the information on the measured and/or estimated biosignals can be provided to the user through the display formed on the front side of the biosignal measurement apparatus 100, or may be stored in a storage unit (not shown) provided in the biosignal measurement apparatus.

[Vehicle Steering Wheel]

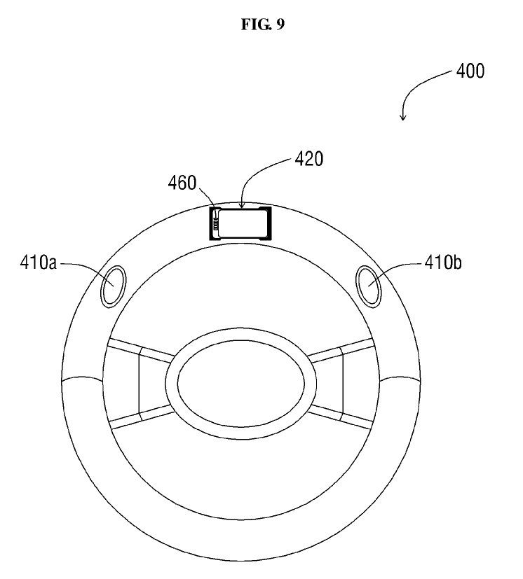

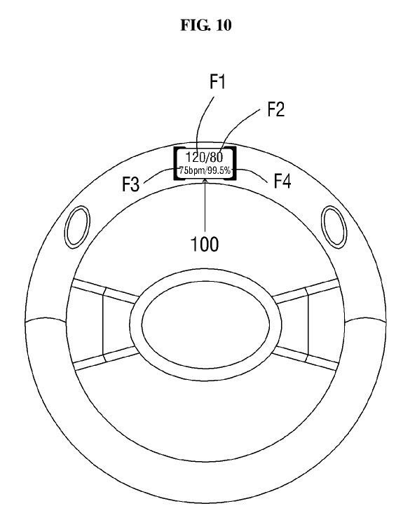

FIGS. 9 and 10 illustrate an embodiment in which the biosignal measurement apparatus 100 can be operated as mounted on a vehicle steering wheel 400. When biosignals of a user are measured using the vehicle steering wheel 400, sensors for measuring the biosignals can be disposed where a driver's hands are naturally positioned at the time of driving the vehicle so that the driver may measure the biosignals more conveniently. Thus, the vehicle steering wheel 400 according to one embodiment can be configured to form sensors or sensor modules for measuring biosignals where the left and right hands of the driver are placed at the time of driving the vehicle so that various biosignals of the driver are measured by the sensors or sensor modules. According to the embodiment shown in FIG. 9, the vehicle steering wheel 400 can be configured to form a first switching electrode 410a for an ECG sensor in the upper left part where the left hand of the driver is positioned at the time of driving the vehicle, and to form a second switching electrode 410b for an ECG sensor in the upper right part where the right hand of the driver is positioned at the time of driving the vehicle. Here, the term, “switching electrodes” refer to electrodes to which the functions of the electrodes A, B formed in the biosignal measurement apparatus are transferred. Further, a PPG sensor or sensor module (light sensor or sensor module) for measuring PPG and/or SpO₂ signals can be provided in at least one of the positions where the first switch electrode 410a and the second switch electrode 410b are formed. Meanwhile, a mounting part 420 for mounting the biosignal measurement apparatus 100 can be provided in an area of the vehicle steering wheel 400. The mounting part 420 can be formed in a shape similar to that of the mounting part 220 of the watch-type counterpart object 200 shown in FIG. 4 so that the biosignal measurement apparatus 100 can be inserted from the front of the steering wheel. In addition, the mounting part can be configured as a variety of commonly known connecting means for sliding and inserting the biosignal measurement apparatus from one side, fixing the biosignal measurement apparatus using magnetic force, or the like.

FIG. 10 shows that the biosignal measurement apparatus 100 can be mounted on the vehicle steering wheel 400 shown in FIG. 9. When the biosignal measurement apparatus 100 is mounted on the vehicle steering wheel 400 as shown in FIG. 10, an ECG signal of the driver can be measured by the ECG sensor (i.e., the first switching electrode 410a and the second switching electrode 410b) formed in the vehicle steering wheel 400, and PPG and SpO₂ signals of the driver can be measured by the PPG sensor formed in the vehicle steering wheel 400. Further, it is possible to estimate blood pressure of the user in real time using these signals. Thus, when it is recognized that the biosignal measurement apparatus 100 according to one embodiment is mounted on the vehicle steering wheel 400 through the electrical contact 160, the biosignal measurement apparatus 100 can be configured to deactivate the sensors formed in the biosignal measurement apparatus 100 and instead activate the sensors formed in the vehicle steering wheel 400 to measure ECG, PPG and SpO₂ signals of the driver and estimate blood pressure of the driver in real time based on these biosignals.

For example, as shown in FIG. 10, when the driver puts a finger of the left hand into contact with the first switching electrode 410a formed in the vehicle steering wheel 400 and puts a finger of the right hand into contact with the second switching electrode 410b while the biosignal measurement apparatus 100 is mounted on the vehicle steering wheel 400, an ECG signal of the user can be measured by the first switching electrode 410a and the second switching electrode 410b. Further, PPG and SpO₂ signals of the driver can be measured together by the PPG sensor formed in the vehicle steering wheel 400.

Meanwhile, in the case of the vehicle steering wheel 400 shown in FIGS. 9 and 10, biosignals (ECG signal) of the driver are generally measured while the first switching electrode 410a and the second switching electrode 410b are in contact with the driver's fingers. Thus, when it is recognized that the biosignal measurement apparatus 100 according to one embodiment is mounted on the vehicle steering wheel 400 through the electrical contact 160, the biosignal measurement apparatus 100 can be configured to assume that an ECG signal is measured while different fingers of the driver are in contact with the first switching electrode 410a and the second switching electrode 410b, and to accordingly correct the biosignal (ECG signal) based on this assumption so that the biosignal can be measured more accurately.

The information on the measured and/or estimated biosignals can be provided to the user through the display 170 formed in the biosignal measurement apparatus 100, through a navigation system of the vehicle, or the like (for example, information on numerical values of systolic blood pressure F1, diastolic blood pressure F2, pulse F3, oxygen saturation level F4 and the like may be displayed through the display 170 of the biosignal measurement apparatus 100 as shown in FIG. 10), or can be transmitted to and stored in a storage unit (not shown) provided in the biosignal measurement apparatus 100 or the vehicle, through an electrical connection between the electrical contact 160 and an electrical contact 460 of the steering wheel 400. Further, it can also be used for estimating blood pressure of the driver in real time by a control unit (not shown) formed in the biosignal measurement apparatus 100 or the vehicle.

[Finger Rest of an Oxygen Saturation Level Measurement Device]

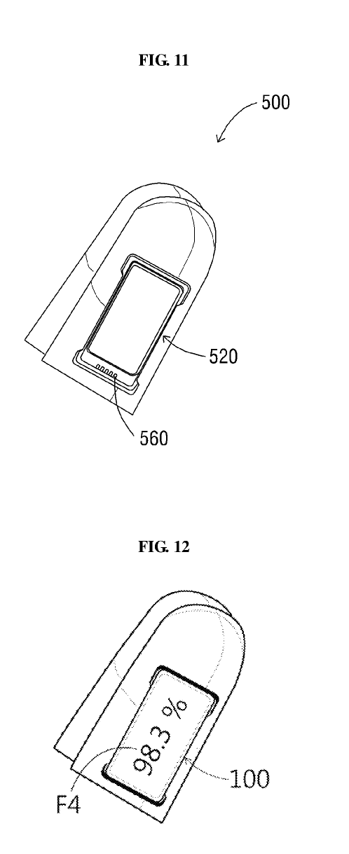

FIGS. 11 and 12 illustrate an embodiment in which the biosignal measurement apparatus 100 can be mounted on a finger rest 500 of an oxygen saturation level measurement device. The oxygen saturation level measurement device is a device for measuring an oxygen saturation level (SpO₂) in a human body, i.e., the content of oxygen present in hemoglobin among various components of blood, using a light sensor. Thus, when it is recognized that the biosignal measurement apparatus 100 is mounted on the finger rest 500 of the oxygen saturation level measurement device as the electrical contact 160 is in contact with an electrical contact 560 formed in the finger rest 500 of the oxygen saturation level measurement device, the biosignal measurement apparatus 100 can be configured to only activate a function for measuring an oxygen saturation level by the PPG sensor or sensor module and deactivate other functions.

For example, as shown FIG. 12, when the biosignal measurement apparatus 100 is operated as mounted on a mounting part 520 formed in the finger rest 500 of the oxygen saturation level measurement device, an SpO₂ signal of a user can be measured by the PPG sensor formed on the rear side of the biosignal measurement apparatus 100, and information on the measured SpO₂ signal F4 can be provided to the user through the display of the biosignal measurement apparatus 100, or can be stored in a storage unit (not shown) provided in the biosignal measurement apparatus.

Further, when a plurality of electrodes of the ECG sensor or sensor module formed in the biosignal measurement apparatus 100 are put into contact with the user's body while the biosignal measurement apparatus 100 is mounted on the finger rest 500 of the oxygen saturation measurement device, it is also possible to measure an ECG signal of the user. Thus, when it is determined that the plurality of electrodes of the ECG sensor formed in the biosignal measurement apparatus 100 are put into contact with the user's body while the biosignal measurement apparatus 100 is mounted on the finger rest 500 of the oxygen saturation measurement device (e.g., when a finger inserted to the finger rest 500 of the oxygen saturation measurement device is in contact with the first electrode A formed on the rear side of the biosignal measurement apparatus 100, and another finger is in contact with the second electrode B formed in the biosignal measurement apparatus 100), the biosignal measurement apparatus 100 can also be configured to further activate a function for measuring an ECG signal by the ECG sensor so that the ECG signal of the user can be measured.

As described above, the biosignal measurement apparatus 100 can be configured to be mounted on various counterpart objects and appropriately activate necessary biosignal measurement functions according to the counterpart objects onto which it is mounted. Further, the biosignal measurement apparatus 100 can be configured to specify the manner of measuring biosignals according to the counterpart objects and accordingly correct the measured biosignals, thereby more accurately acquiring the biosignals generated from the respective body parts.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A biosignal measurement apparatus operable in combination with a plurality of counterpart objects, comprising:
an ECG sensor having a first electrode formed on a rear side of the biosignal measurement apparatus, and a second electrode formed apart from the first electrode;
at least one PPG sensor having a light emitter configured to generate light to be irradiated to a body part, and a light receiver configured to receive light irradiated by the light emitter and reflected by the body part; and
an apparatus electrical contact electrically connectable to an electrical contact formed in a counterpart object to be combined,
wherein the biosignal measurement apparatus is configured to measure at least one of electrocardiogram (ECG), photoplethysmogram (PPG), and oxygen saturation level (SpO₂) biosignals of a user using at least one of the ECG sensor and the PPG sensor, and
wherein the biosignal measurement apparatus is configured to recognize a type of the combined counterpart object based on an electrical connection between the apparatus electrical contact of the biosignal measurement apparatus and the electrical contact formed in the counterpart object, to activate functions for measuring measurable biosignals according to the type of the combined counterpart object, and to correct values of the measured biosignals according to the type of the combined counterpart object.

2. The biosignal measurement apparatus of claim 1, further comprising:
a display formed on a front side of the biosignal measurement apparatus and configured to display information to the user, the display having a measurement area configured to measure biosignals of the user,
wherein red (R) subpixels configured to form red light and infrared (IR) subpixels configured to form infrared light are included in a pixel structure formed in the measurement area of the display,
wherein the red (R) subpixels and infrared (IR) subpixels form the light emitter of one of the at least one PPG sensor, and
wherein the light receiver of the PPG sensor whose light emitter is formed with the red (R) subpixels and infrared (IR) subpixels is formed in the measurement area of the display.

3. The biosignal measurement apparatus of claim 1, further comprising:
at least one additional electrode for the ECG sensor spaced apart from the first electrode and the second electrode.

4. The biosignal measurement apparatus of claim 1, wherein the biosignal measurement apparatus is configured to be operable as mounted on a watch-type counterpart object, and
wherein, when the biosignal measurement apparatus is mounted on the watch-type counterpart object, the biosignal measurement apparatus is configured to activate a function for measuring an ECG signal by the ECG sensor and a function for measuring PPG and SpO₂ signals by the PPG sensor.

5. The biosignal measurement apparatus of claim 4, wherein, when the biosignal measurement apparatus is mounted on the watch-type counterpart object, the biosignal measurement apparatus is configured to correct the ECG signal by assuming that the ECG signal is measured while the first electrode is in contact with a wrist of the user and the second electrode is in contact with a finger of the user.

6. The biosignal measurement apparatus of claim 5, wherein the biosignal measurement apparatus is configured to estimate blood pressure of the user based on the measured ECG, PPG and SpO₂ signals.

7. The biosignal measurement apparatus of claim 1, wherein the biosignal measurement apparatus is configured to be operable as mounted on a necklace-type counterpart object, and
wherein, when the biosignal measurement apparatus is mounted on the necklace-type counterpart object, the biosignal measurement apparatus is configured to activate a function for measuring an ECG signal by the ECG sensor and a function for measuring PPG and SpO₂ signals by the PPG sensor.

8. The biosignal measurement apparatus of claim 7, wherein, when the biosignal measurement apparatus is mounted on the necklace-type counterpart object 300, the biosignal measurement apparatus is configured to correct the ECG signal by assuming that the ECG signal is measured while the first electrode and the second electrode are in contact with different fingers of the user.

9. The biosignal measurement apparatus of claim 8, wherein the biosignal measurement apparatus is configured to estimate blood pressure of the user based on the measured ECG, PPG and SpO₂ signals.

10. The biosignal measurement apparatus of claim 7, wherein at least one auxiliary electrode is disposed on a necklace string of the necklace-type counterpart object, and
wherein, when the biosignal measurement apparatus is mounted on the necklace-type counterpart object, the biosignal measurement apparatus is configured to measure an ECG signal of the user based on signals from at least two electrodes contacting a body of the user, among the first electrode and the second electrode formed in the biosignal measurement apparatus and the at least one auxiliary electrode formed on the necklace string of the necklace-type counterpart object.

11. The biosignal measurement apparatus of claim 10, wherein, when the biosignal measurement apparatus is mounted on the necklace-type counterpart object, the biosignal measurement apparatus is configured to correct the ECG signal by assuming that signals from the auxiliary electrode formed on the necklace string are generated while the auxiliary electrode is in contact with a neck of the users, and signals from the first electrode and the second electrode of the biosignal measurement apparatus are generated while the first electrode and the second electrode are in contact with fingers of the user.

12. The biosignal measurement apparatus of claim 11, wherein the biosignal measurement apparatus is configured to estimate blood pressure of the user based on the measured ECG, PPG and SpO₂ signals.

13. The biosignal measurement apparatus of claim 1, wherein the biosignal measurement apparatus is configured to be operable as mounted on a vehicle steering wheel, and
wherein, when the biosignal measurement apparatus is mounted on the vehicle steering wheel, the ECG sensor and the PPG sensor formed in the biosignal measurement apparatus are configured to be deactivated, and a steering wheel ECG sensor (i) disposed on the steering wheel, (ii) configured to measure an ECG signal, and (iii) having a first switching electrode and a second switching electrode, and a steering wheel PPG sensor (i) disposed on the steering wheel and (ii) configured to measure PPG and SpO₂ signals, are configured to be activated.

14. The biosignal measurement apparatus of claim 13, wherein, when the biosignal measurement apparatus is mounted on the vehicle steering wheel, the biosignal measurement apparatus is configured to correct the ECG signal by assuming that the ECG signal is measured while the first switching electrode and the second switching electrode are in contact with different fingers of the user.

15. The biosignal measurement apparatus of claim 14, wherein the biosignal measurement apparatus is configured to estimate blood pressure of the user based on the measured ECG, PPG and SpO₂ signals.

16. The biosignal measurement apparatus of claim 1, wherein the biosignal measurement apparatus is configured to be operable as mounted on a finger rest of an oxygen saturation level measurement device, and
wherein, when the biosignal measurement apparatus is mounted on the finger rest of the oxygen saturation level measurement device, the biosignal measurement apparatus is configured to activate a function for measuring a SpO₂ signal by the PPG sensor.

17. The biosignal measurement apparatus of claim 16, wherein, when one or more of the electrodes of the ECG sensor formed in the biosignal measurement apparatus are put into contact with a body of the user while the biosignal measurement apparatus is mounted on the finger rest of the oxygen saturation measurement device, the ECG sensor is configured to measure an ECG signal.
